(19) United States
(12) Patent Application Publication
(10) Pub. No.: US 2021/0251549 A1
(43) Pub. Date: Aug. 19, 2021

(54) METHOD OF VISUALIZATION OF THE PATIENT'S BODY SURFACE AND DETERMINING THE COORDINATES OF ECG ELECTRODES DURING NON-INVASIVE ELECTROPHYSIOLOGICAL MAPPING OF THE HEART

(71) Applicant: LIMITED LIABILITY COMPANY "COMPUTER MODELING SYSTEMS", ST. PETERSBURG (RU)

(72) Inventors: Aleksandr Georgievich Petrov, St. Petersburg (RU); Mikhail Petrovich Chmelevsky, St. Petersburg (RU)

(21) Appl. No.: 16/973,164

(22) PCT Filed: Jun. 6, 2019
(86) PCT No.: PCT/RU2019/000406

§ 371 Date: Dec. 8, 2020

(30) Foreign Application Priority Data
Jun. 9, 2018 (RU) 2018121418

(51) Int. Cl. A61B 5/318; A61B 5/00; A61B 5/28

(52) U.S. Cl. CPC A61B 5/318; A61B 5/0037; A61B 5/28; A61B 5/0064; A61B 5/0044

(57) ABSTRACT

A three-dimensional model of the patients body surface of 360 degrees with the coordinates of ECG electrodes is formed via a computer program according to data of three-dimensional photo-scanning of the patients body surface from above and data of three-dimensional photo-scanning of the patients back imprint. Position of ECG electrodes on the body surface relative to the surface of the heart is determined via computer simulation and combining a three-dimensional model of the body surface of 360 degrees, obtained during three-dimensional photo-scanning, and a three-dimensional model of the heart and the inner surface of the chest, obtained during CT or MRI procedure. Combining a three-dimensional model of the body surface of 360 degrees, obtained during three-dimensional photo-scanning, and a three-dimensional model of the heart and the inner surface of the chest, obtained during CT or MRI procedure, is performed on the basis of the specific anatomic characteristics of the patient's body surface and the inner surface of the chest, which are deemed to be unchanged for a specified time period, or on the basis of combining three markers, visible during three-dimensional photo-scanning and during CT or MRI procedure, or on the basis of a single system of coordinates for three-dimensional photo-scanning and CT or MRI procedure. The invention implementation results in improvement of the technique of visualization of the patients body surface.

### TECHNICAL FIELD

The invention relates to medicine, in particular, to cardiology and functional diagnostics, and can be used during the diagnostic procedure of non-invasive electrophysiological mapping of the heart.

### PRIOR ART

Currently, CT and MM data are used for visualization of the patient's body surface and determining the coordinates of ECG electrodes during non-invasive electrophysiological mapping of the heart [1, 2, 3, 4, 5].

The above-mentioned method of visualization of the patient's body surface and determining the coordinates of ECG electrodes has a number of disadvantages:
high cost of the procedure
significant radiation exposure to the patient (CT) and associated therewith restrictions concerning frequency of use (in particular [5] p. 96 “ . . . the use of MSCT limits the possibility of widespread clinical use of the technique due to the increasing radiation exposure to the patient.”)
restriction on the use of MRI for patients with implanted cardiac pacemakers
the complexity of performing CT and MRI of the torso in heavy patients due to the need of long-term (more than 30 seconds) breath holding
restrictions, associated with the presence of relative and absolute medical contraindications to performing CT and MRI (pregnancy, allergy to contrast agents, the presence of metal implants, etc.)

The invention aims to solve these problems by achieving the simplicity of visualization of the patient's body surface with determining the coordinates of ECG electrodes and an unlimited repetition rate of non-invasive electrophysiological mapping of the patient's heart.

### SUMMARY OF THE INVENTION

The method of the present disclosure has the following distinctive features compared to the existing method of visualization of the patient's body surface and determining the coordinates of ECG electrodes:
CT or MRI procedure is used one time only to obtain a three-dimensional model of the heart and the inner surface of the chest.
The existing method of obtaining a three-dimensional model of the patient's body surface and determining the coordinates of ECG electrodes based on data, obtained from CT or MRI of the chest, is substituted with a method of obtaining a three-dimensional model of the patient's body surface and determining the coordinates of ECG electrodes based on data of three-dimensional photo-scanning of the body surface.
Combining a three-dimensional model of the body surface with the coordinates of ECG electrodes, obtained on the basis of three-dimensional photo-scanning data, and a three-dimensional model of the heart and the inner surface of the chest, obtained from CT or MRI data using three-dimensional simulation software.

The method of the present disclosure exploits a system of disposable multi-contact ECG electrodes, applied vertically around the entire circumference of the torso, as well as an installation of three-dimensional photo-scanning with a system of the back imprint and software for three-dimensional simulation of holotopy, which includes:
A software module for conversion of CT and MRI data in a polygon three-dimensional model, which ensures three-dimensional computer simulation of the heart and the inner surface of the chest.
A software module for three-dimensional computer simulation of the results of photo-scanning, which ensures computer simulation of the patient's body surface and generating a polygon model with determining the coordinates of ECG electrodes.
A software module for three-dimensional computer simulation of holotopy, which ensures determining the relative position of the heart surface and the body surface with ECG electrodes and creating a general polygon model of the heart holotopy with the coordinates of ECG electrodes.
A software interface module, which ensures data formatting to be used during the procedure of non-invasive electrophysiological mapping of the heart and storing in the patient's health information database.

FIG. 1 illustrates the sequence of operations to be implemented in time according to the method of the present disclosure.

The first step 1 includes performing CT or MRI of the patient's chest, as a result of which files are generated in the DICOM format, or the use of images, obtained on the basis of previously performed CT or MRI of the patient's chest. Moreover, the CT and MRI procedures are obligatory in case of a known and recorded event that results to changes in size, geometry or inner structure of the heart and the inner surface of the chest. The first step may not be associated with mapping of the heart and can be performed by other reasons. The first step can be performed using the methods of ultrasound heart examination [6], rotational X-ray study [7], electrical impedance tomography [8], intraoperative transesophageal echocardiography [6]

The second step 2 includes three-dimensional simulation of the heart and the inner surface of the chest by processing files in the DICOM format, obtained as a result of the first step implementation, and generating a polygon model of the heart and the inner surface of the chest. The second step and subsequent operations may arise out of the decision to perform non-invasive mapping of the heart.

The third step 3 is provided by applying ECG electrodes around the circumference of the patient's torso. FIG. 2 illustrates an example of the torso with surface ECG electrodes (1) being applied,

The fourth step 4 ensures placement of the patient in a lying position with surface ECG electrodes in an installation of three-dimensional photo-scanning with the back imprint forming system and implementation of three-dimensional photo-scanning of the body surface with ECG electrodes applied from above. FIG. 3 illustrates a lateral view of the patient placed in a lying position (2) in an installation of three-dimensional photo-scanning (3) with an imprint system (4). FIG. 4 illustrates a front view of the patient placed in a lying position (2) in an installation of three-dimensional photo-scanning (3) with an imprint system (4). FIG. 5 illustrates a photograph of a model for an installation of three-dimensional photo-scanning (3) of the patient (2).

The fifth step 5 includes three-dimensional photo-scanning of the imprint, formed by the back of the patient with ECG electrodes (5) after lifting the patient to a sitting position (6). FIG. 6 illustrates a lateral view of the patient placed in a sitting position (6) in an installation of three-dimensional photo-scanning (3) with an imprint system, formed by the patient's back with ECG electrodes (5) being applied. FIG. 7 illustrates a front view of the patient placed in a sitting position (6) in an installation of three-dimensional photo-scanning (3) with the imprint, formed by the patient's back with ECG electrodes (5) being applied.

The sixth step 6 is implemented via software for three-dimensional simulation based on data, obtained from digital photographs of the patient's body surface with ECG electrodes, patient's back imprint with ECG electrodes, combining and generating a general three-dimensional model of the body surface with ECG electrodes, determining the coordinates of ECG electrodes. FIGS. 8 and 9 illustrate an example of a three-dimensional model of the patient's body with ECG electrodes applied from above, formed via software based on processing of data, obtained from digital photo cameras. FIGS. 10, 11, 12 illustrate an example of software operation for three-dimensional simulation of the hand. FIG. 10 illustrates an example of software operation for three-dimensional simulation of the hand upper surface. FIG. 11 illustrates an example of software operation for three-dimensional simulation of the palm print. FIG. 12 illustrates an example of software operation for combining three-dimensional models of the hand upper surface and the palm into a single three-dimensional model of the hand surface, presented in the form of a polygon mesh.

The seventh step 7 is implemented via software operation for three-dimensional simulation of the patient's heart holotopy and creating a general polygon model of the heart relative to the body surface with the coordinates of ECG electrodes using a polygon model of the heart and the inner surface of the chest, obtained on the basis of CT or MRI data, and a polygon model of the patient's body surface with the coordinates of ECG electrodes, obtained on the basis of three-dimensional photo-scanning data.

The eighth step 8 ensures formatting of data on a general polygon model of the heart relative to the body surface with the coordinates of ECG electrodes to be subsequently used during the procedure of non-invasive electrophysiological mapping of the heart and storing data in the patient's health information database (10).

The cycle of operations 3, 4, 5, 6, 7, 8, 9 can be repeated without limitations by the rate of frequency.

### BRIEF DESCRIPTION OF THE FIGURES

FIG. 1 illustrates the sequence of operations.

FIG. 2 illustrates an example of applying ECG electrodes (1) around the circumference of the patient's torso.

FIG. 3 illustrates a lateral view of the patient placed in a lying position (2) in an installation of three-dimensional photo-scanning (3) with an imprint system (4).

FIG. 4 illustrates a front view of the patient placed in a lying position (2) in an installation of three-dimensional photo-scanning (3) with an imprint system (4).

FIG. 5 illustrates a photograph of a model for an installation of three-dimensional photo-scanning (3) of the patient (2).

FIG. 6 illustrates a lateral view of the patient placed in a sitting position (6) in an installation of three-dimensional photo-scanning (3) with an imprint system, formed by the patient's back with ECG electrodes (5).

FIG. 7 illustrates a front view of the patient placed in a sitting position (6) in an installation of three-dimensional photo-scanning (3) with an imprint system, formed by the patient's back with ECG electrodes (5).

FIG. 8 illustrates an example of a three-dimensional model of the patient's body with ECG electrodes applied from above, formed via software based on processing of data, obtained from digital photo cameras.

FIG. 9 illustrates an example of a three-dimensional model of the patient's body with ECG electrodes applied from above, with a polygon mesh representation, formed via software based on processing of data, obtained from digital photo cameras.

FIG. 10 illustrates an example of software operation for three-dimensional simulation of the hand upper surface.

FIG. 11 illustrates an example of software operation for three-dimensional simulation of the palm print.

FIG. 12 illustrates an example of software operation for combining three-dimensional models of the hand upper surface and the palm into a single three-dimensional model of the hand surface, presented in the form of a polygon mesh.

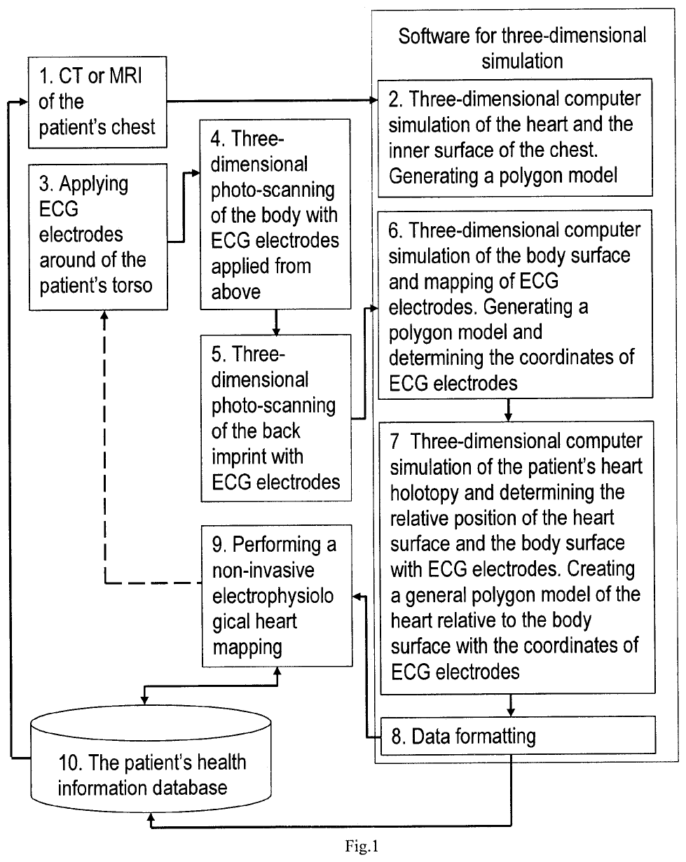

### DETAILED DESCRIPTION OF THE INVENTION

The disclosed method of visualization of the patient's body surface with determining the coordinates of ECG electrodes during non-invasive electrophysiological mapping of the heart is based on three-dimensional photo-scanning and computer simulation and can be applied practically by those skilled in the art.

The operations on the method testing have been carried out to confirm the possibility of the present disclosure implementation.

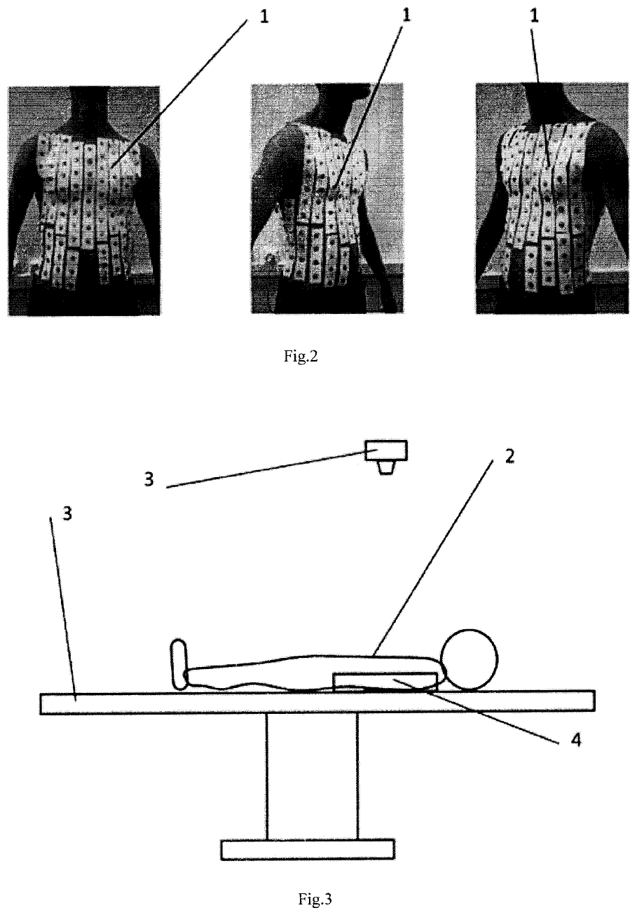

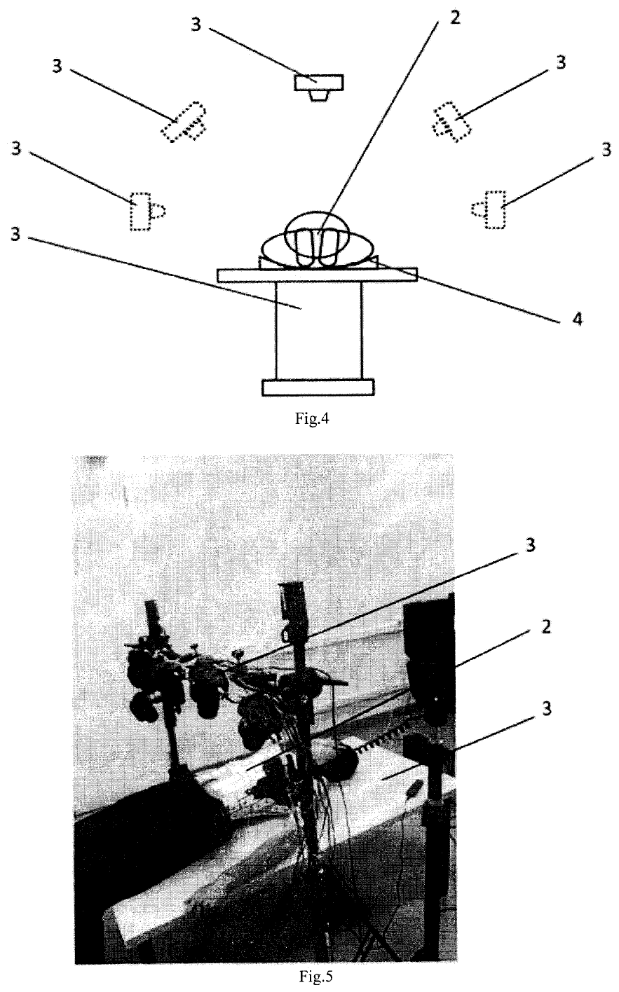

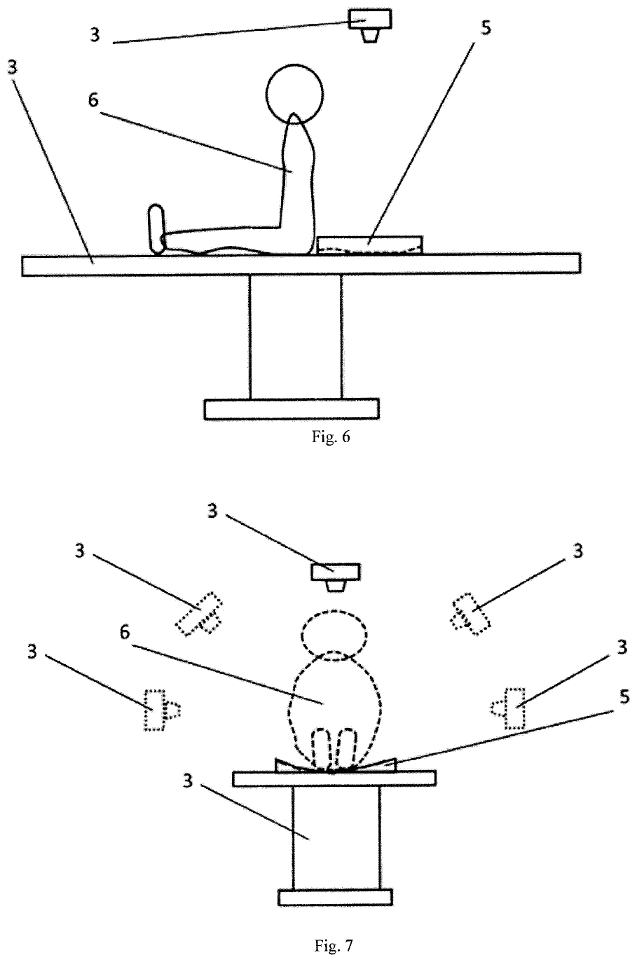

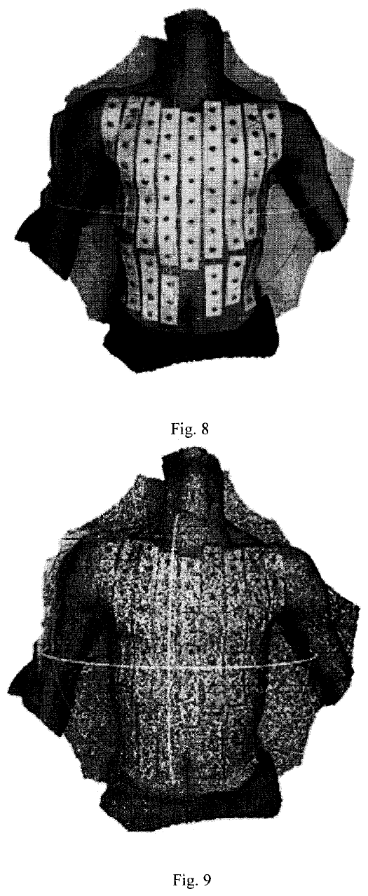

FIGS. 2, 5, 8, and 9 illustrate operations on testing three-dimensional photo-scanning of the patient with a model of surface ECG electrodes (1) and operations on computer simulation of the results of three-dimensional photo-scanning of the patient's body surface with ECG electrodes applied from above.

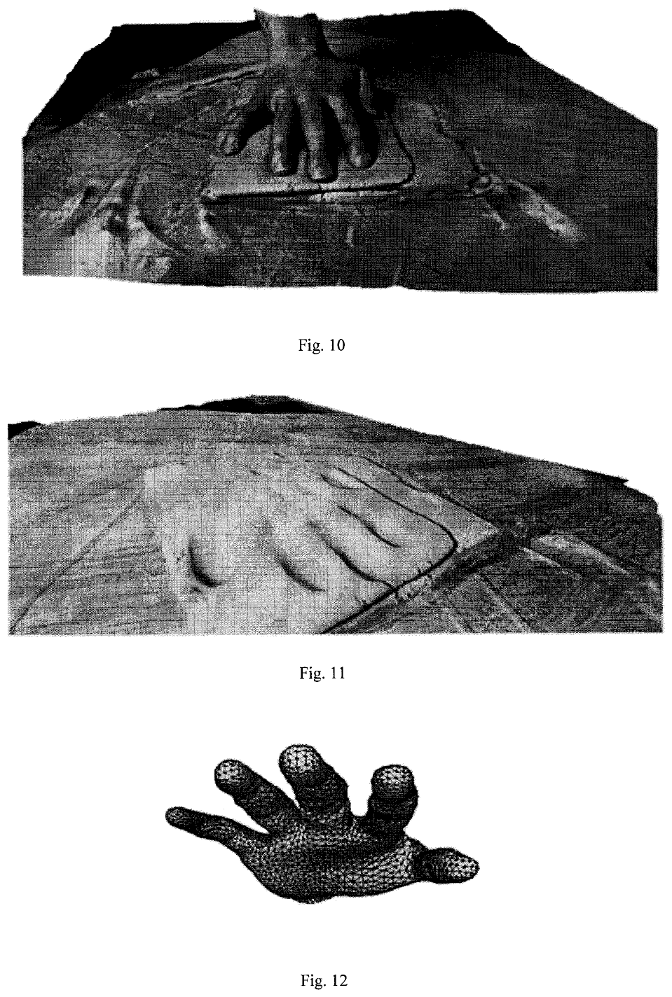

FIGS. 10, 11 and 12 illustrate, by the example of the hand, the results of testing three-dimensional photo-scanning with an imprint system and software operation for combining three-dimensional models of the hand upper surface and the palm into a single three-dimensional model of the hand surface, presented in the form of a polygon mesh.

The imprint obtaining system uses material that meets sanitary and hygienic rules and standards, applicable in health care facilities, and having plasticity and deformation characteristics under the patient's torso weight, as well as shape memory, sufficient to place the patient in a sitting position and perform the imprint photo-scanning, with relatively short restoration time for the material's suitability for re-print.

The imprint system is the original method for obtaining a three-dimensional model of the patient's body surface of 360 degrees. Three-dimensional simulation of the patient's body surface of 360 degrees can be performed by other methods as well. Provision can be made that the invention includes obtaining a three-dimensional model of the patient's body surface of 360 degrees by other methods, presented in the claims.

Three-dimensional simulation of the patient's heart holotopy is performed by a computer program with algorithms for scaling and comparing two three-dimensional polygon models, having common surfaces for comparison based on the specific anatomic characteristics of the patient's body surface and the inner surface of the chest, which are deemed to be unchanged for a specified time period. To compare the two models, CT or MRI imaging and three-dimensional photo-scanning of the patient's body surface are performed at inhale.

The invention has been described with respect to advantageous embodiments. Upon the above-mentioned detailed description has been read and understood, modifications and variations can be assumed. Provision can be made that the invention includes all possible modifications and variations, since they fall within the scope of the appended claims or equivalents thereof.

### BIBLIOGRAPHY

1. Patent RU2409313C2, A. Sh. Revishvili, V. V. Kalinin, A. V. Kalinin. Method of non-invasive electrophysiological heart examination

2. Patent RU2417051C2, A. Sh. Revishvili, V. V. Kalinin, A. V. Kalinin. Method of non-invasive electrophysiological heart examination

3. Patent RU2435518C2, A. Sh. Revishvili, V. V. Kalinin, A. V. Kalinin. Method of non-invasive electrophysiological heart examination

4. M. P. Chmelevsky, S. V. Zubarev, M. A. Budanova. Non-invasive electrophysiological mapping in the diagnostics of ventricular arrhythmias. Pub. FSBI “NMRC named after V.A. Almazov” of the Russian Ministry of Health. Translational medicine, 2 (5) 2015

5. Zubarev S. V., Chmelevsky M. P., Budanova M. A., Trukshina M. A., Lyubimtseva T. A., Lebedeva V. K., Lebedev D. S. Non-invasive electrophysiological mapping and the effect of cardiac resynchronization therapy: the importance of the position of left ventricular electrode. Pub. FSBI “NMRC named after V.A. Almazov” of the Russian Ministry of Health. Translational medicine, 3 (3) 2016

6. M. A. Saidova. Three-dimensional echocardiography: yesterday, today, tomorrow. Consilium Medicum. 2006; 5: 127-132. Consilium Medicum portal: http://con-med.ru/magazines/consilium_medicum/consilium_medicum-05-2006/trekhmernaya_ekhokardiografiya_vchera_segodnya_zavtra/

7. N. M. Fedotov, A. I. Oferkin, A. A. Shelupanov Method of integrating the data of rotational X-ray study and electrical location for visualization of anatomic structures of the heart and surgical instrument. Reports by TSUCSR, No. 2 (26), part 2, December 2012. UDC 621.386.8; 616-079.2

8. E. V. Miroshnichenko. Electrical impedance computed tomography. ILPI Research and Engineering Center “Technocentre” TSUR. Proceedings by TSUR Thematic issue. MIS-2004 Hardware and software for medical diagnostics and therapy. UDC-612.014.42: 573 (043.3)

1. Method of visualization of the patient's body surface with the coordinates of ECG electrodes during non-invasive electrophysiological mapping of the heart wherein:
a three-dimensional model of the patient's body surface of 360 degrees with the coordinates of ECG electrodes is formed via a computer program according to data of three-dimensional photo-scanning of the patient's body surface from above and data of three-dimensional photo-scanning of the patient's back imprint,
position of ECG electrodes on the body surface relative to the surface of the heart is determined via computer simulation and combining a three-dimensional model of the body surface of 360 degrees, obtained during three-dimensional photo-scanning, and a three-dimensional model of the heart and the inner surface of the chest, obtained during CT or MRI procedure.
Combining a three-dimensional model of the body surface of 360 degrees, obtained during three-dimensional photo-scanning, and a three-dimensional model of the heart and the inner surface of the chest, obtained during CT or MRI procedure, is performed on the basis of the specific anatomic characteristics of the patient's body surface and the inner surface of the chest, which are deemed to be unchanged for a specified time period.

2. Method as claimed in claim 1, characterized in that a three-dimensional model of the patient's body surface of 360 degrees with the coordinates of ECG electrodes is formed via a computer program according to data of three-dimensional photo-scanning of 360 degrees of the patient's body surface without the use of the back imprint.

3. Method as claimed in claim 1, characterized in that combining a three-dimensional model of the body surface of 360 degrees, obtained during three-dimensional photo-scanning, and a three-dimensional model of the heart and the inner surface of the chest, obtained during CT or MRI procedure, is performed using at least three markers, visible during three-dimensional photo-scanning and during CT or MRI procedure.

4. Method as claimed in claim 1, characterized in that combining a three-dimensional model of the body surface of 360 degrees, obtained during three-dimensional photo-scanning, and a three-dimensional model of the heart and the inner surface of the chest, obtained during CT or MRI procedure, is performed within a single system of coordinates for three-dimensional photo-scanning and CT or MRI procedure.

5. Method as claimed in claim 1, characterized in that a three-dimensional model of the heart and the inner surface of the chest, is obtained based on data of ultrasound heart examination, rotational X-ray study, electrical impedance tomography, intraoperative transesophageal echocardiography.
